(19) United States
(12) Patent Application Publication
(10) Pub. No.: US 2023/0337986 A1
(43) Pub. Date: Oct. 26, 2023

(54) POSITIONING METHOD, POSITIONING APPARATUS, ELECTRONIC DEVICE, AND STORAGE MEDIUM

(71) Applicant: SUNRISE TECHNOLOGIES CO., LTD., Beijing (CN)

(72) Inventors: Kexin Xu, Beijing (CN); Tongshuai Han, Beijing (CN)

(21) Appl. No.: 18/005,218

(22) PCT Filed: May 12, 2021
(86) PCT No.: PCT/CN2021/093246

§ 371 Date: Jan. 12, 2023

(30) Foreign Application Priority Data
Jul. 14, 2020 (CN) 202010677650.5

(51) Int. Cl. A61B 5/00

(52) U.S. Cl. CPC A61B 5/70

(57) ABSTRACT

The embodiments of the present disclosure provide a positioning method, a positioning apparatus, an electronic device and a storage medium. The method includes: acquiring a measurement region of an object; determining a positioning portion of the object according to the measurement region; and positioning the positioning portion to position the measurement region and ensure that a measurement posture meets a predetermined condition.

### CROSS REFERENCE TO RELATED APPLICATION(S)

This application is a Section 371 National Stage Application of International Application No. PCT/CN2021/093246, filed on May 12, 2021, entitled “POSITIONING METHOD, POSITIONING APPARATUS, ELECTRONIC DEVICE, AND STORAGE MEDIUM”, which claims priority to Chinese Patent Application No. 202010677650.5, filed on Jul. 14, 2020, the disclosures of which are incorporated herein by reference in their entireties.

### TECHNICAL FIELD

The present disclosure relates to a field of measurement technology, and in particular to a positioning method, a positioning apparatus, an electronic device, and a storage medium.

### BACKGROUND

An optical detection method has characteristics of rapidness, non-invasiveness, and multidimensional information, etc., and is generally adopted to detect a tissue element. The tissue element includes blood sugar, hemoglobin, and fat, etc.

In the related art, it is required to position a measurement region in order to obtain spectral data of the measurement region. The measurement region is generally positioned directly by using a positioning apparatus. During a positioning process, the positioning apparatus may be in contact with the measurement region.

However, it is found that at least the following problems exist in the related art. When the measurement region is positioned by using the related art, a measurement condition may not be ensured, thereby affecting a detection accuracy.

### SUMMARY

According to an aspect of the present disclosure, a positioning method is provided, including: acquiring a measurement region of an object; determining a positioning portion of the object according to the measurement region; and positioning the positioning portion to position the measurement region and ensure that a measurement posture meets a predetermined condition.

According to the embodiments of the present disclosure, the determining a positioning portion of the object according to the measurement region includes: determining the positioning portion of the object according to a tissue structure relative position of the object, a feature condition of the object and the measurement region of the object.

According to the embodiments of the present disclosure, the positioning the positioning portion to position the measurement region and ensure that a measurement posture meets a predetermined condition includes: positioning the positioning portion through a positioning apparatus to position the measurement region and ensure that the measurement posture meets the predetermined condition.

According to the embodiments of the present disclosure, the positioning apparatus includes a supporting part and a positioning part; and a relationship between the positioning part and the supporting part satisfies at least one of: the positioning part being arranged on the supporting part; the positioning part being connected to the supporting part; and the positioning part being embedded in the supporting part; and the positioning the positioning portion through the positioning apparatus to position the measurement region and ensure that the measurement posture meets the predetermined condition includes: carrying the measurement region and/or the positioning portion through the supporting part; and positioning the positioning portion through the positioning part to position the measurement region and ensure that the measurement posture meets the predetermined condition.

According to the embodiments of the present disclosure, a region corresponding to the measurement region is a hollow region.

According to the embodiments of the present disclosure, the positioning part includes a knuckle part configured to position at least one of a little finger, a ring finger, a middle finger and an index finger; and the positioning part is embedded in the supporting part; the measurement region is a hand region, and the positioning portion includes at least one of the little finger, the ring finger, the middle finger and the index finger; and the carrying the measurement region and/or the positioning portion through the supporting part includes: carrying the measurement region and the positioning portion through the supporting part.

According to the embodiments of the present disclosure, the hand region includes a region on an outer side of a proximal knuckle of the index finger; and the positioning portion includes the little finger, the ring finger, the middle finger and the index finger.

According to the embodiments of the present disclosure, the positioning part includes a knuckle part configured to position at least one of a little finger, a ring finger, a middle finger and an index finger and an elbow joint part configured to position an elbow joint; and the positioning part is arranged on the supporting part or embedded in the supporting part; the measurement region includes an arm region; and the positioning portion includes the elbow joint, and at least one of the little finger, the ring finger, the middle finger and the index finger; and the carrying the measurement region and/or the positioning portion through the supporting part includes: carrying the measurement region and the positioning portion through the supporting part.

According to the embodiments of the present disclosure, the arm region includes a forearm extensor side region; and the positioning portion includes the little finger, the ring finger, the middle finger, the index finger and the elbow joint.

According to the embodiments of the present disclosure, the positioning part further includes a carpus part configured to position a carpus; and the positioning portion further includes the carpus.

According to the embodiments of the present disclosure, the positioning part includes an eye corner part configured to position an eye corner and a nose part configured to position a nose; and the positioning part is connected to the supporting part; the measurement region is a head region; and the positioning portion includes the eye corner and the nose; and the carrying the measurement region and/or the positioning portion through the supporting part includes: carrying the measurement region through the supporting part.

According to the embodiments of the present disclosure, the head region includes a forehead region.

According to another aspect of the present disclosure, a positioning apparatus is provided, including a supporting part and a positioning part, a relationship between the positioning part and the supporting part satisfies at least one of: the positioning part being arranged on the supporting part; the positioning part being connected to the supporting part; and the positioning part being embedded in the supporting part; the supporting part is configured to carry a measurement region and/or a positioning portion of an object; and the positioning part is configured to position the positioning portion to position the measurement region and ensure that a measurement posture meets a predetermined condition.

According to the embodiments of the present disclosure, the supporting part includes a hollow region corresponding to the measurement region.

According to the embodiments of the present disclosure, a positional relationship between the positioning portion and the measurement region meets a predetermined tissue structure relative position.

According to the embodiments of the present disclosure, the positioning part includes a knuckle part configured to position at least one of a little finger, a ring finger, a middle finger and an index finger; and the positioning part is embedded in the supporting part; the measurement region is a hand region; and the positioning portion includes at least one of the little finger, the ring finger, the middle finger and the index finger; and the supporting part is configured to carry the measurement region and the positioning portion.

According to the embodiments of the present disclosure, the hand region includes a region on an outer side of a proximal knuckle of the index finger; and the positioning portion includes the little finger, the ring finger, the middle finger and the index finger.

According to the embodiments of the present disclosure, the positioning part includes a knuckle part configured to position at least one of a little finger, a ring finger, a middle finger and an index finger and an elbow joint part configured to position an elbow joint; and the positioning part is arranged on the supporting part or embedded in the supporting part; the measurement region is an arm region, and the positioning portion includes the elbow joint, and at least one of the little finger, the ring finger, the middle finger and the index finger; and the supporting part is configured to carry the measurement region and the positioning portion.

According to the embodiments of the present disclosure, the arm region includes a forearm extensor side; and the positioning portion includes the little finger, the ring finger, the middle finger, the index finger and the elbow joint.

According to the embodiments of the present disclosure, the positioning part further includes a carpus part configured to position a carpus; and the positioning portion further includes the carpus.

According to the embodiments of the present disclosure, the positioning part includes an eye corner part configured to position an eye corner and a nose part configured to position a nose; and the positioning part is connected to the supporting part; the measurement region is a head region, and the positioning portion includes the eye corner and the nose; and the supporting part is configured to carry the measurement region.

According to the embodiments of the present disclosure, the head region includes a forehead region.

According to another aspect of the present disclosure, an electronic device is provided, including: one or more processors; a memory configured to store one or more programs; the one or more programs, when executed by the one or more processors, cause the one or more processors to implement the method described above.

According to another aspect of the present disclosure, a computer-readable storage medium having computer programs stored thereon is provided, the programs, when executed by a processor, implement the method described above.

According to another aspect of the present disclosure, a computer program product including computer programs is provided, the computer programs, when executed by a processor, implement the method described above.

### BRIEF DESCRIPTION OF THE DRAWINGS

FIG. 1 is a flowchart of a positioning method in the embodiments of the present disclosure;

FIG. 2 is a schematic diagram of knuckles of an index finger in the embodiments of the present disclosure;

FIG. 3 is a schematic diagram of a forearm in the embodiments of the present disclosure;

FIG. 4 is a flowchart of another positioning method in the embodiments of the present disclosure;

FIG. 5 is a schematic structural diagram of a positioning apparatus in the embodiments of the present disclosure;

FIG. 6 is a schematic structural diagram of another positioning apparatus in the embodiments of the present disclosure;

FIG. 7 is a schematic diagram of an application of a positioning apparatus in the embodiments of the present disclosure;

FIG. 8 is a schematic structural diagram of yet another positioning apparatus in the embodiments of the present disclosure;

FIG. 9 is a schematic diagram of an application of another positioning apparatus in the embodiments of the present disclosure;

FIG. 10 is a schematic structural diagram of still another positioning apparatus in the embodiments of the present disclosure;

FIG. 11 is a schematic diagram of an application of yet another positioning apparatus in the embodiments of the present disclosure;

FIG. 12 is a flowchart of a method of preparing a positioning apparatus in the embodiments of the present disclosure;

FIG. 13 is a flowchart of a method of preparing another positioning apparatus in the embodiments of the present disclosure; and

FIG. 14 is a schematic structural diagram of an electronic device in the embodiments of the present disclosure.

### DETAILED DESCRIPTION OF EMBODIMENTS

The present disclosure will be further described in detail below in conjunction with the accompanying drawings and the embodiments. It may be understood that the specific embodiments described here are merely for explaining the present disclosure, rather than limiting the present disclosure. Various features recited in the embodiments may be combined to form a plurality of optional solutions. In addition, it should be noted that, for ease of description, only some structures, rather than all structures related to the present disclosure are shown in the accompanying drawings.

During a process of implementing the concept of the present disclosure, the inventors found that at least the following problems exist in the related art. First, since a positioning apparatus may be in contact with a measurement region and thus affect a state of the measurement region during a positioning process, a measurement condition may not be ensured. Second, since there is no positioning feature in the measurement region, the measurement condition may not be ensured. Since the measurement condition may not be ensured, a positioning accuracy may not be ensured, thereby a detection accuracy may also be affected. The measurement region may refer to a region used for a non-invasive detection of tissue element.

In order to ensure the measurement condition, improve the positioning accuracy and thus improve the detection accuracy, it is necessary to ensure that the positioned measurement region remains in a predetermined state. In order to solve the above-mentioned problems, it is possible to position a positioning portion to indirectly position the measurement region. The positioning portion may refer to a portion for positioning. According to the embodiments of the present disclosure, since it is necessary to ensure that the positioned measurement region remains in a predetermined state, it is required that a change of a skin status of the positioning portion may not affect a skin status of the measurement region.

Based on the above, the positioning portion is required to meet the following conditions. First, a distance between the positioning portion and the measurement region is within a predetermined distance range. Second, the change of the skin status of the positioning portion affects the skin status of the measurement region as small as possible. Third, the positioning portion has an obvious positioning feature. With regard to the second condition, it may be understood from another perspective that, a spectral variation of the measurement region caused by the change of the positioning portion is within a predetermined variation range. The first and second conditions described above may be attributed to the fact that a positional relationship between the positioning portion and the measurement region meets a predetermined tissue structure relative position. The positioning portion having an obvious positioning feature is to facilitate an accurate positioning.

According to the embodiments of the present disclosure, due to a change of a measurement posture may lead to a change of a relative position between the measurement region and the positioning portion, and the change of the measurement posture may cause the skin status of the measurement region to change, thus it is required for an accurate positioning of the measurement region to take an accurate positioning of the measurement posture as a premise. The positioning portion determined based on the above-mentioned conditions may ensure that the measurement posture meets a predetermined condition. Here, the measurement posture meeting the predetermined condition may be understood as that after each positioning is completed, a change of a relative position of skin tissues in the measurement region is within a predetermined position change range.

The above-mentioned positioning manner is based on a following principle that due to an object has six degrees of freedom in space, including degrees of freedom of movement along X-axis, Y-axis and Z-axis and degrees of freedom of rotation around the above-mentioned three coordinate axes, thus the six degrees of freedom are required to be restricted to completely position a target object. In order to position the measurement region on the object, it is required to restrict the above-mentioned six degrees of freedom of the measurement region. The six degrees of freedom of the measurement region may be restricted by fixing the positioning portion. The above-mentioned contents will be described below in conjunction with specific embodiments.

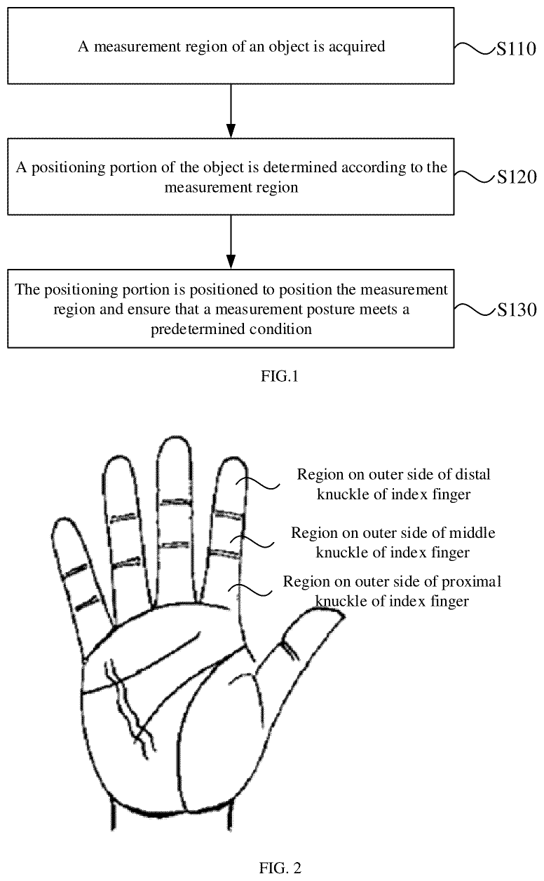

FIG. 1 is a flowchart of a positioning method provided by the embodiments of the present disclosure. The embodiment may be applicable to ensure the measurement condition so as to improve the positioning accuracy and thereby improving the detection accuracy. As shown in FIG. 1, the method specifically includes the following operations.

In operation S110, a measurement region of an object is acquired.

According to the embodiments of the present disclosure, the object may refer to a limb of a creature. The measurement region may refer to a region used for a non-invasive tissue element measurement. The measurement region may include a hand region, an arm region or a head region. For example, the hand region may include a region on an outer side of a proximal knuckle of an index finger. The arm region may include a forearm extensor side region. The head region may include a forehead region. The non-invasive tissue element detection determines a concentration of a tissue element according to spectral data acquired from a measurement position in the measurement region.

In operation S120, a positioning portion of the object is determined according to the measurement region.

According to the embodiments of the present disclosure, after the measurement region of the object is determined, it is required to determine the positioning portion of the object according to the measurement region. That is, the positioning portion corresponding to the measurement region may be determined according to a tissue structure relative position of the object, a feature condition of the object and the measurement region of the object. The feature condition may refer to an obvious positioning feature of the positioning portion. The positioning feature may include a hand knuckle, a limb joint, or a facial portion, etc.

The tissue structure relative position may be understood as follows. A positional relationship between the positioning portion and the measurement region meets a predetermined tissue structure relative position such that a change of a skin status of the positioning portion affects a skin status of the measurement region as small as possible and a distance between the positioning portion and the measurement region is within a predetermined distance range. It may be understood from another perspective that the tissue structure relative position is such that a spectral variation of the measurement region caused by a change of the positioning portion is within a predetermined variation range and a distance between the positioning portion and the measurement region is within a predetermined distance range. The change of the skin status may include a skin deformation and/or an internal change of tissue structure, etc.

For example, if the measurement region is the arm region, the positioning portion meeting the tissue structure relative position with the arm region may be an elbow joint and the hand knuckle. For another example, if the measurement region is the hand region, the positioning portion meeting the tissue structure relative position with the hand region may be the hand knuckle. For still another example, if the measurement region is the head region, the positioning portion meeting the tissue structure relative position with the head region may be the facial portion.

In operation S130, the positioning portion is positioned to position the measurement region and ensure that a measurement posture meets a predetermined condition.

According to the embodiments of the present disclosure, the positioning of the measurement region may be achieved by positioning the positioning portion. The positioning portion determined as mentioned above may ensure that the measurement posture meets the predetermined condition. Here, the measurement posture meeting a predetermined condition may be understood as that, after each positioning is completed, a change of a relative position of skin tissues in the measurement region is within a predetermined position change range. A specific value of the predetermined position change range may be set according to actual situations, which is not limited here.

According to the technical solution of the embodiments of the present disclosure, the positioning portion is positioned to position the measurement region and ensure that the measurement posture meets the predetermined condition, so that the measurement condition is ensured, and thereby the positioning accuracy is improved. With the improvement of the positioning accuracy, a subsequent measurement error may be effectively controlled, and thereby the detection accuracy may be improved.

According to the embodiments of the present disclosure, determining the positioning portion of the object according to the measurement region may include the following operations.

The positioning portion of the object is determined according to the tissue structure relative position of the object, a feature condition of the object and the measurement region of the object.

According to the embodiments of the present disclosure, the tissue structure relative position may be understood as follows. A positional relationship between the positioning portion and the measurement region meets a predetermined tissue structure relative position such that a change of a skin status of the positioning portion affects a skin status of the measurement region as small as possible and a distance between the positioning portion and the measurement region is within a predetermined distance range. It may be understood from another perspective that the tissue structure relative position is such that a spectral variation of the measurement region caused by a change of the positioning portion is within a predetermined variation range and a distance between the positioning portion and the measurement region is within a predetermined distance range. The positioning feature may include a hand knuckle, a limb joint, or a facial portion, etc.

Exemplarily, the measurement region may be a region on an outer side of a proximal knuckle of an index finger, forearm extensor side region or a forehead region. If the measurement region is the region on the outer side of the proximal knuckle of the index finger, the positioning portion may include a little finger, a ring finger, a middle finger and the index finger. If the measurement region is the forearm extensor side region, the positioning portion may include the little finger, the ring finger, the middle finger, the index finger, a palm and an elbow joint, and may further include a carpus. If the measurement region is the forehead region, the positioning portion may include eye corners and a nose. It should be noted that the measurement region may be other regions. Accordingly, the positioning portion may be other portions.

According to the embodiments of the present disclosure, the positioning the positioning portion to position the measurement region and ensure the measurement posture to meet the predetermined condition may include the following operations.

The positioning portion is positioned through a positioning apparatus to position the measurement region and ensure that the measurement posture meets the predetermined condition.

According to the embodiments of the present disclosure, the positioning apparatus may be used to position the positioning portion, so that the positioning of the measurement region may be achieved indirectly. Since the positioning portion may ensure that the measurement posture meets the predetermined condition, it may ensure that the measurement posture meets the predetermined condition during an entire positioning process. For example, the positioning apparatus may include a supporting part and a positioning part. The measurement region and/or the positioning portion may be carried by the supporting part. The positioning portion may be positioned by the positioning part so as to achieve the positioning of the measurement region and ensure that the measurement posture meets the predetermined condition.

As stated above, the positioning portion is fixed by the positioning apparatus so as to achieve the positioning of the measurement region and ensure that the measurement posture meets the predetermined condition, so that the measurement condition is ensured, and thereby the positioning accuracy may be improved. With the improvement of the positioning accuracy, a subsequent measurement error may be effectively controlled, and thereby the detection accuracy is improved.

According to the embodiments of the present disclosure, the positioning apparatus includes the supporting part and the positioning part. A relationship between the positioning part and the supporting part satisfies at least one of: the positioning part being arranged on the supporting part, the positioning part being connected to the supporting part, and the positioning part being embedded in the supporting part.

The positioning the positioning portion by using the positioning apparatus to position the measurement region and ensure the measurement posture to meet the predetermined condition may include the following operations. The measuring region and/or the positioning portion are/is carried by the supporting part. The positioning portion is positioned by the positioning part so as to position the measurement region and ensure that the measurement posture meets the predetermined condition.

According to the embodiments of the present disclosure, the positioning apparatus may include the supporting part and the positioning part. The supporting part may be used to carry the measurement region and/or the positioning portion. A material of the supporting part and the positioning part may be nylon, resin, acrylic or metal, etc.

The relationship between the positioning part and the supporting part satisfies at least one of: the positioning part being arranged on the supporting part, the positioning part being connected to the supporting part, and the positioning part being embedded in the supporting part. The positioning part being arranged on the supporting part may refer to that the positioning part exists in a form of a protrusion on the supporting part. The positioning part being connected to the supporting part may refer to that the positioning part and the supporting part are connected through a connector. The positioning part being embedded in the supporting part may refer to that the positioning part is embedded inside the supporting part. The above-mentioned positional relationship may be determined according to the measurement region and the positioning portion.

The positioning of the measurement region achieved by using the positioning apparatus includes placing the measurement region and/or the positioning portion on the supporting part, and positioning the positioning portion by the positioning part, so as to indirectly position the measurement region. That is, the positioning of the positioning portion is achieved by placing the positioning portion on the positioning part, and the positioning of the measurement region is achieved by the positioning of the positioning portion. In addition, since the positioning apparatus may be applied to different measurement regions, it has a strong practicability. Moreover, a preparation cost is low and an operation is simple and easy.

According to the embodiments of the present disclosure, a region corresponding to the measurement region is a hollow region.

According to the embodiments of the present disclosure, in order to prevent the positioning apparatus from contacting the measurement region and affecting the state of the measurement region during the positioning process, the region corresponding to the measurement region may be provided as a hollow region, so that when the measurement region is placed on the supporting part, it may ensure that the skin status of the measurement region is affected as less as possible.

In addition, a region corresponding to the positioning portion may be provided as completely hollow, partially hollow or non-hollow, which may be set according to actual situations and is not limited here. The positioning of the measurement region may be achieved indirectly by positioning the positioning portion using the positioning part. That is, the positioning of the positioning portion is achieved by placing the positioning portion on the positioning part, and the positioning of the measurement region is achieved through the positioning of the positioning portion.

According to the embodiments of the present disclosure, the positioning part includes a knuckle part configured to position at least one of the little finger, the ring finger, the middle finger and the index finger. The positioning part is embedded in the supporting part. The measurement region is the hand region. The positioning portion includes at least one of the little finger, the ring finger, the middle finger and the index finger. The carrying the measurement region and/or the positioning portion by the supporting part may include the following operations. The measurement region and the positioning portion are carried by the supporting part.

According to the embodiments of the present disclosure, the knuckle part may be configured to position at least one of the little finger, the ring finger, the middle finger and the index finger. The knuckle part may be understood as a component prepared based on at least one of the little finger, the ring finger, the middle finger and the index finger. A preparation method may include 3D printing or machining.

According to the embodiments of the present disclosure, the hand region includes the region on the outer side of the proximal knuckle of the index finger. The positioning portion includes the little finger, the ring finger, the middle finger and the index finger.

According to the embodiments of the present disclosure, the knuckle part may be configured to position the little finger, the ring finger, the middle finger and the index finger. The knuckle part may be understood as a component prepared based on first two knuckles (i.e., a distal knuckle and a middle knuckle) of the little finger, the ring finger, the middle finger and the index finger. The knuckles of fingers may be divided into distal knuckles, middle knuckles and proximal knuckles based on distances from the knuckles to the heart. Knuckles of the index finger may refer to FIG. 2. As shown in FIG. 2, a schematic diagram of knuckles of the index finger is shown. The region on the outer side of the knuckles of the index finger is shown in FIG. 2.

In order to prevent the positioning apparatus from contacting the region on the outer side of the proximal knuckle of the index finger (i.e., the measurement region) and affecting the state of the measurement region during the positioning process, a region corresponding to the measurement region on the supporting part may be provided as a hollow region. On the supporting part, in addition to the region corresponding to the measurement region on the outer side of the proximal knuckle of the index finger being provided as hollow, regions corresponding to other regions on the outer side of the proximal knuckle of the index finger may be provided as partially hollow, completely hollow or non-hollow, which may be set according to actual situations, and is not limited here.

After the four fingers are fixed by the knuckle part, five degrees of freedom (i.e., three degrees of freedom of movement, and degrees of freedom of rotation around the X-axis and Z-axis) among the six degrees of freedom are restricted. On this basis, the remaining degree of freedom (i.e., the degree of freedom of rotation around the Y-axis) may be restricted by placing the palm on the supporting part. Since the six degrees of freedom of the region on the outer side of the proximal knuckle of the index finger are restricted, the region on the outer side of the proximal knuckle of the index finger is therefore positioned. As stated above, the positioning of the region on the outer side of the proximal knuckle of the index finger is achieved by the positioning of the little finger, the ring finger, the middle finger and the index finger through the knuckle part and the fixing of the palm through the supporting part.

According to the embodiments of the present disclosure, the positioning part includes the knuckle part configured to position at least one of the little finger, the ring finger, the middle finger and the index finger, and an elbow joint part configured to position the elbow joint. The positioning part is arranged on the supporting part or embedded in the supporting part. The measurement region includes the arm region. The positioning portion includes the elbow joint, and at least one of the little finger, the ring finger, the middle finger and the index finger. The carrying the measurement region and/or the positioning portion by the supporting part may include the following operations. The measurement region and the positioning portion are carried by the supporting part.

According to the embodiments of the present disclosure, the knuckle part may be configured to position at least one of the little finger, the ring finger, the middle finger and the index finger. The knuckle part may be understood as a component prepared based on at least one of the little finger, the ring finger, the middle finger and the index finger. The elbow joint part may be configured to position the elbow joint. The elbow joint part may be understood as a component prepared after a position of the elbow joint is determined based on a length of the arm and a feature of the elbow joint. A method of preparing the knuckle part and the elbow joint part may include 3D printing or machining.

According to the embodiments of the present disclosure, the arm region includes the forearm extensor side region. The positioning portion includes the little finger, the ring finger, the middle finger, the index finger and the elbow joint.

According to the embodiments of the present disclosure, the knuckle part may be configured to position the little finger, the ring finger, the middle finger and the index finger. The knuckle part may be understood as the component prepared based on the first two knuckles of the little finger, the ring finger, the middle finger and the index finger.

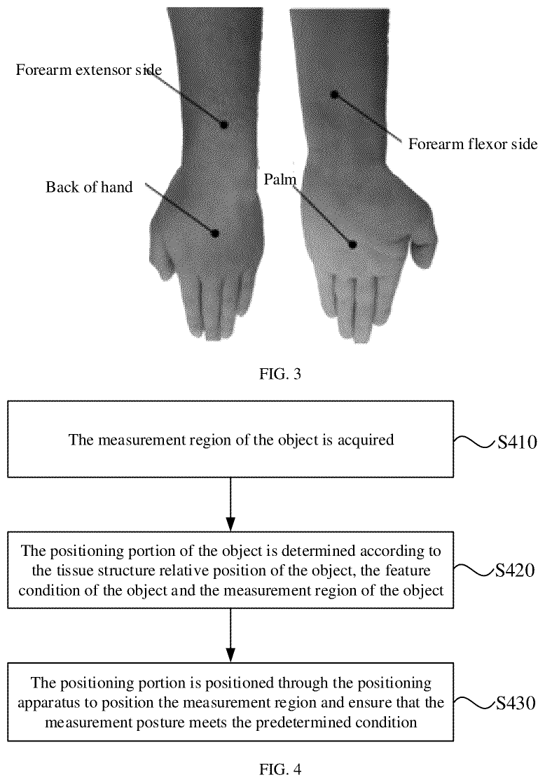

In order to prevent the positioning apparatus from contacting the forearm extensor side region (that is, the measurement region) and affecting the state of the measurement region during the positioning process, a region corresponding to the measurement region on the supporting part may be provided as a hollow region. The knuckle part may be embedded in the supporting part, and the elbow joint may be arranged on the supporting part. The forearm extensor side may refer to FIG. 3. FIG. 3 is a schematic diagram of a forearm, and a forearm extensor side and a forearm flexor side are shown in FIG. 3.

According to the embodiments of the present disclosure, on the supporting part, in addition to the region corresponding to the measurement region on the forearm extensor side being provided as hollow, regions corresponding to other regions on the forearm extensor side may be provided as partially hollow, completely hollow or non-hollow, which may be set according to actual situations, and is not limited here.

After the four fingers are fixed by the knuckle part and the elbow joint is fixed by the elbow joint part, the six degrees of freedom are restricted. As stated above, the positioning of the forearm extensor side region is achieved by the positioning of the little finger, the ring finger, the middle finger and the index finger through the knuckle part in cooperation with the positioning of the elbow joint through the elbow joint part.

According to the embodiments of the present disclosure, the positioning part further includes a carpus part configured to position a carpus. The positioning portion further includes the carpus.

According to the embodiments of the present disclosure, the carpus part may be understood as a component prepared based on a position of the carpus. A method of preparing the carpus part may include 3D printing or machining.

The six degrees of freedom are restricted after the four fingers are fixed by the knuckle part and the elbow joint is fixed by the elbow joint part. However, due to a particularity of a wrist, the carpus may be fixed by using the carpus part so as to better determine a rotation angle of the arm and further improve the positioning accuracy. As stated above, the positioning of the forearm extensor side region is achieved by the positioning of the little finger, the ring finger, the middle finger and the index finger through the knuckle part in cooperation with the positioning of the carpus through the carpus part and the positioning of the elbow joint through the elbow joint part.

According to the embodiments of the present disclosure, the positioning part includes an eye corner part configured to position an eye corner and a nose part configured to position a nose. The positioning part is connected to the supporting part. The measurement region is the head region. The positioning portion includes the eye corner and the nose. The carrying the measurement region and/or the positioning portion by the supporting part may include the following operations. The measurement region is carried by the supporting part.

According to the embodiments of the present disclosure, the eye corner part may be configured to position the eye corner. The eye corner part may be understood as a component prepared based on the eye corner. The nose part may be configured to position the nose. The nose part may be understood as a component prepared based on the nose. A preparation method may include 3D printing or machining.

According to the embodiments of the present disclosure, the head region includes the forehead region.

According to the embodiments of the present disclosure, in order to prevent the positioning apparatus from contacting the forehead region (i.e., the measurement region) and affecting the state of the measurement region during the positioning process, a region corresponding to the measurement region on the supporting part may be provided as a hollow region. On the supporting part, in addition to the region corresponding to the measurement region on the forehead being provided as hollow, regions corresponding to other regions on the forehead may be provided as partially hollow, completely hollow or non-hollow, which may be set according to actual situations, and is not limited here.

After the eye corner is fixed by the eye corner part and the nose is fixed by the nose part, the six degrees of freedom are restricted. Due to the six degrees of freedom of the forehead region are restricted, the positioning of the forehead region is achieved. As stated above, the positioning of the forehead region is achieved by the positioning of the eye corner through the eye corner part in cooperation with the positioning of the nose through the nose part.

According to the embodiments of the present disclosure, in a process of positioning the forehead by using the positioning apparatus, the eye is required to in a lightly closed state because blinking may cause a positioning error.

According to the embodiments of the present disclosure, a material of the supporting part and the positioning part may be nylon, resin, acrylic or metal.

FIG. 4 is a flowchart of another positioning method provided by the embodiments of the present disclosure. The embodiment may be applicable to ensure the measurement condition so as to improve the positioning accuracy and thereby improving the detection accuracy. As shown in FIG. 4, the method specifically includes the following operations.

In operation S410, the measurement region of the object is acquired.

In operation S420, the positioning portion of the object is determined according to the tissue structure relative position of the object, the feature condition of the object and the measurement region of the object.

In operation S430, the positioning portion is positioned through the positioning apparatus to position the measurement region and ensure that the measurement posture meets the predetermined condition.

According to the technical solution of the embodiments of the present disclosure, the positioning portion is positioned through the positioning apparatus to position the measurement region and ensure that the measurement posture meets the predetermined condition, so that the measurement condition is ensured, and thereby the positioning accuracy is improved. With the improvement of the positioning accuracy, the subsequent measurement error may be effectively controlled, and thereby the detection accuracy is improved. In addition, since the corresponding positioning apparatus is prepared according to the measurement region, it has a strong practicability. Moreover, the preparation cost is low and the operation is simple and easy.

FIG. 5 is a schematic structural diagram of a positioning apparatus provided by the embodiments of the present disclosure. The embodiment may be applicable to ensure the measurement condition so as to improve the positioning accuracy and thereby improving the detection accuracy. As shown in FIG. 5, the positioning apparatus may include a supporting part 1 and a positioning part 2. A relationship between the positioning part 2 and the supporting part 1 satisfies at least one of: the positioning part 2 being arranged on the supporting part 1, the positioning part 2 being connected to the supporting part 1, and the positioning part 2 being embedded in the supporting part 1. A structure and a working principle thereof will be described below with reference to the accompanying drawings.

The supporting part 1 is configured to carry the measurement region and/or the positioning portion of the object.

The positioning part 2 is configured to position the positioning portion to position the measurement region and ensure that measurement posture meets the predetermined condition.

According to the embodiments of the present disclosure, for descriptions of the supporting part 1 and the positioning part 2, reference may be made to the corresponding portions above, which will not be repeated here.

According to the technical solution of the embodiments of the present disclosure, the positioning portion is positioned through the positioning apparatus to position the measurement region and ensure that the measurement posture meets the predetermined condition, so that the measurement condition is ensured, and thereby the positioning accuracy is improved. With the improvement of the positioning accuracy, the subsequent measurement error may be effectively controlled, and thereby the detection accuracy is improved. In addition, since the corresponding positioning apparatus is prepared according to the measurement region, it has a strong practicability. Moreover, the preparation cost is low and the operation is simple and easy.

According to the embodiments of the present disclosure, as shown in FIG. 5, the supporting part includes a hollow region corresponding to the measurement region.

According to the embodiments of the present disclosure, as shown in FIG. 5, a rectangular box indicated by reference numeral 11 may represent the hollow region. The rectangular box schematically shows the hollow region, which is merely for the purpose of illustration, and is not intended to limit a shape of the hollow region. Any suitable shape may be adopted for the hollow region.

According to the embodiments of the present disclosure, the positional relationship between the positioning portion and the measurement region meets the predetermined tissue structure relative position.

According to the embodiments of the present disclosure, since the positioning portion is a portion meeting the tissue structure relative position with the measurement region, and the positioning part corresponds to the positioning portion while the hollow region corresponds to the measurement region, therefore, if the positional relationship between the positioning portion and the measurement region meets the predetermined tissue structure relative position, the positional relationship between the positioning part and the hollow region meets the predetermined tissue structure relative position accordingly.

According to the embodiments of the present disclosure, the positioning part 2 includes a knuckle part 20 configured to position at least one of the little finger, the ring finger, the middle finger and the index finger. The positioning part 2 is embedded in the supporting part 1. The measurement region is the hand region. The positioning portion includes at least one of the little finger, the ring finger, the middle finger and the index finger. The supporting part 1 is used to carry the measurement region and the positioning portion.

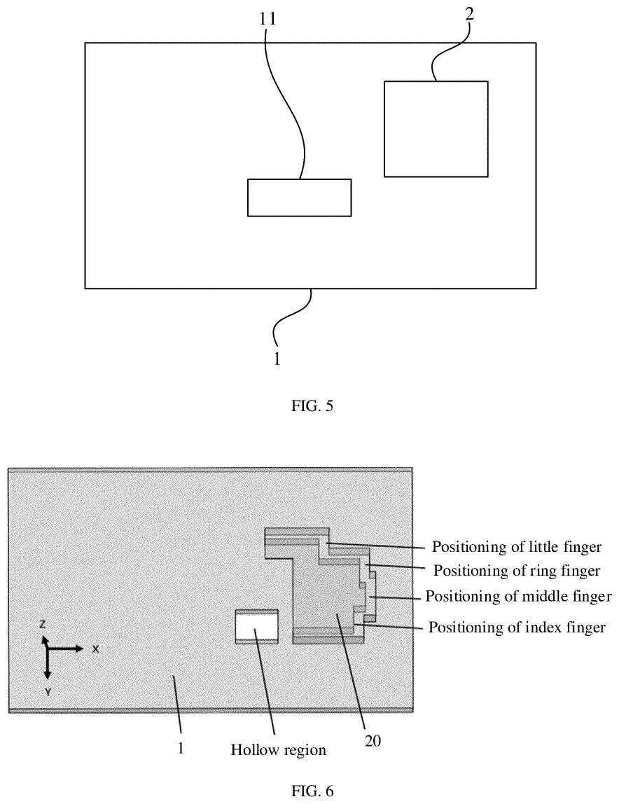

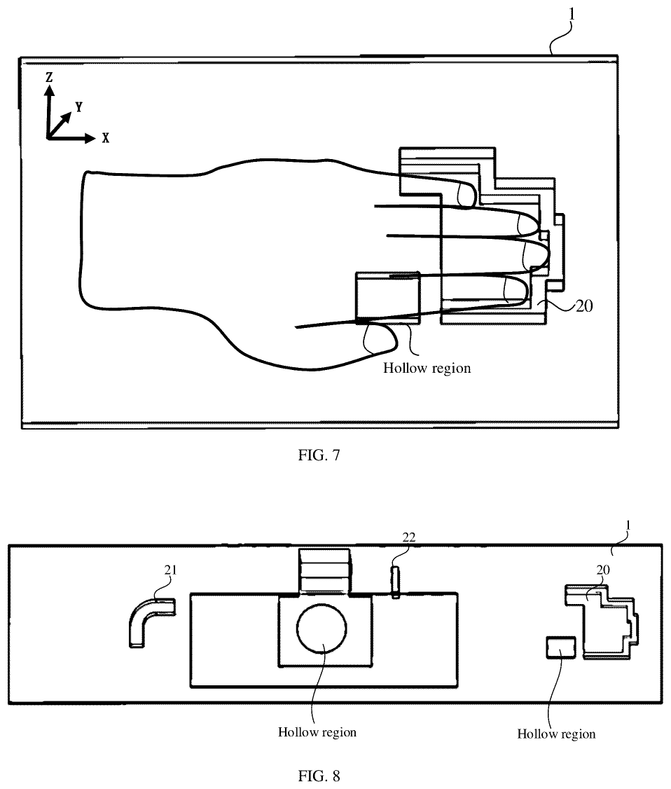

According to the embodiments of the present disclosure, as shown in FIG. 6 and FIG. 7, the hand region includes the region on the outer side of the proximal knuckle of the index finger. The positioning portion includes the little finger, the ring finger, the middle finger and the index finger.

According to the embodiments of the present disclosure, with reference to FIG. 6 and FIG. 7, FIG. 6 is a schematic structural diagram of another positioning apparatus, and FIG. 7 is a schematic diagram of an application of a positioning apparatus. In FIG. 7, five degrees of freedom (i.e., three degrees of freedom of movement, and degrees of freedom of rotation around the X-axis and the Z-axis) among the six degrees of freedom are restricted after the four fingers are fixed by the knuckle part 20. On this basis, the remaining degree of freedom (i.e., the degree of freedom of rotation around the Y-axis) may be restricted by placing the palm on the supporting part 1. Since the six degrees of freedom of the region on the outer side of the proximal knuckle of the index finger are restricted, the region on the outer side of the proximal knuckle of the index finger is positioned.

According to the embodiments of the present disclosure, the positioning part 2 includes the knuckle part 20 configured to position at least one of the little finger, the ring finger, the middle finger and the index finger, and an elbow joint part 21 configured to position the elbow joint. The positioning part 2 is arranged on the supporting part 1 or embedded in the supporting part 1. The measurement region is the arm region. The positioning portion includes the elbow joint, and at least one of the little finger, the ring finger, the middle finger and the index finger. The supporting part 1 is used to carry the measurement region and the positioning portion.

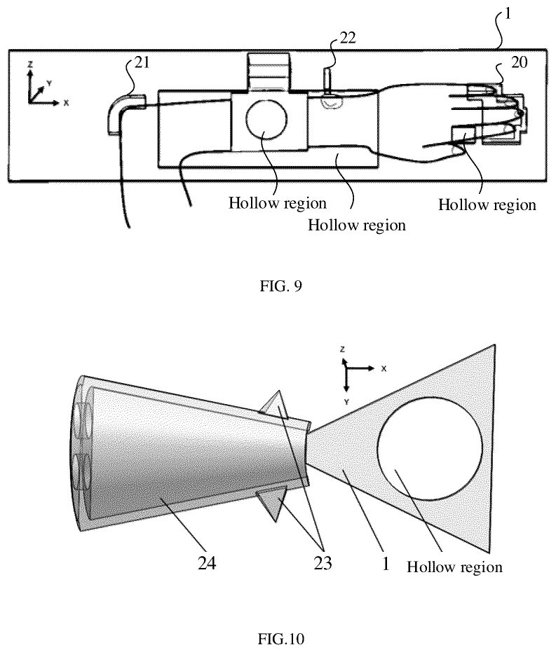

According to the embodiments of the present disclosure, as shown in FIG. 8 and FIG. 9, the arm region includes the forearm extensor side. The positioning portion includes the little finger, the ring finger, the middle finger, the index finger and the elbow joint.

According to the embodiments of the present disclosure, with reference to FIG. 8 and FIG. 9, FIG. 8 is a schematic structural diagram of yet another positioning apparatus, and FIG. 9 is a schematic diagram of an application of another positioning apparatus. In FIG. 9, the six degrees of freedom are restricted after the four fingers are fixed by the knuckle part 20 and the elbow joint is fixed by the elbow joint part 21. As stated above, the positioning of the forearm extensor side region is achieved by the positioning of the little finger, the ring finger, the middle finger and the index finger through the knuckle part 20 in cooperation with the positioning of the elbow joint through the elbow joint part 21. In FIG. 9, on the supporting part 1, in addition to the region corresponding to the measurement region on the forearm extensor side being provided as hollow, regions corresponding to other regions on the forearm extensor side are provided as hollow.

According to the embodiments of the present disclosure, as shown in FIG. 8 and FIG. 9, the positioning part 2 further includes a carpus part 22 configured to position the carpus. The positioning portion further includes the carpus.

According to the embodiments of the present disclosure, as shown in FIG. 6 and FIG. 7, the measurement region further includes the region on the outer side of the proximal knuckle of the index finger.

According to the embodiments of the present disclosure, the positioning part includes the eye corner part configured to position the eye corner and the nose part configured to position the nose. The positioning part is connected to the supporting part. The measurement region is the head region. The positioning portion includes the eye corner and the nose. The supporting part is configured to carry the measurement region.

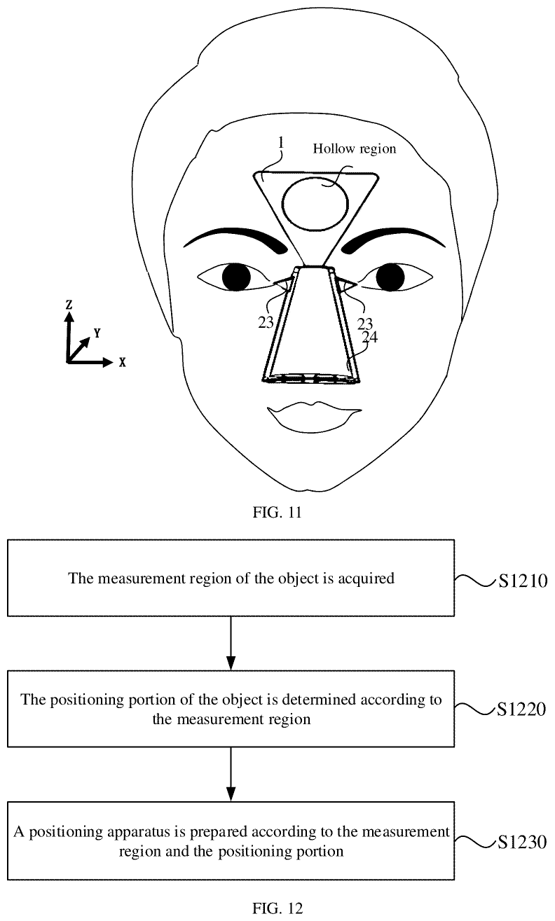

According to the embodiments of the present disclosure, as shown in FIG. 10 and FIG. 11, the head region includes the forehead region.

According to the embodiments of the present disclosure, with reference to FIG. 10 and FIG. 11, FIG. 10 is a schematic structural diagram of still another positioning apparatus, and FIG. 11 is a schematic diagram of an application of yet another positioning apparatus. In FIG. 11, the six degrees of freedom are restricted after the eye corner is fixed by the eye corner part and the nose is fixed by the nose part. Since the six degrees of freedom of the forehead region are restricted, the forehead region is positioned. As stated above, the positioning of the forehead region is achieved by the positioning of the eye corner through the eye corner part 23 in cooperation with the positioning of the nose through the nose part 24.

According to the embodiments of the present disclosure, a material of the supporting part 1 and the positioning part 2 may be nylon, resin, acrylic or metal.

FIG. 12 is a flowchart of a method of preparing a positioning apparatus provided by the embodiments of the present disclosure. The embodiment may be applicable to ensure the measurement condition so as to improve the positioning accuracy and further improving the detection accuracy. As shown in FIG. 12, the method specifically includes the following operations.

In operation S1210, the measurement region of the object is acquired.

In operation S1220, the positioning portion of the object is determined according to the measurement region.

According to the embodiments of the present disclosure, after the measurement region of the object is acquired, the positioning portion corresponding to the measurement region may be determined according to the tissue structure relative position of the object, the feature condition of the object and the measurement region of the object. The tissue structure relative position is such that the change of the skin status of the positioning portion affects the skin status of the measurement region as small as possible and the distance between the positioning portion and the measurement region is within the predetermined distance range. In addition, based on the positioning portion determined above, it may ensure that the measurement posture meets the predetermined condition.

In operation S1230, a positioning apparatus is prepared according to the measurement region and the positioning portion.

According to the embodiments of the present disclosure, after the measurement region and the positioning portion are acquired, the supporting part and the positioning part may be prepared according to the measurement region and the positioning portion. The positioning apparatus may be obtained according to the supporting part and the positioning part.

According to the technical solution of embodiments of the present disclosure, since the corresponding positioning apparatus is prepared according to the measurement region, it has a strong practicability. Moreover, the preparation cost is low and the operation is simple and easy.

According to the embodiments of the present disclosure, the determining the positioning portion of the object according to the measurement region may include the following operations.

The positioning portion of the object is determined according to the tissue structure relative position of the object, the feature condition of the object and the measurement region of the object.

According to the embodiments of the present disclosure, preparing the positioning device according to the measurement region and the positioning portion may include the following operations.

The supporting part and the positioning part are prepared according to the measurement region and the positioning portion so as to obtain the positioning device. The relationship between the positioning part and the supporting part satisfies at least one of: the positioning part being arranged on the supporting part, the positioning part being connect to the supporting part, and the positioning part being embedded in the supporting part.

According to the embodiments of the present disclosure, after the positioning portion and the measurement region are determined, the corresponding positioning part may be prepared according to the positioning portion. The corresponding supporting part may be prepared according to the measurement region and/or the positioning portion. During a preparing process, the relationship between the positioning part and the supporting part satisfies at least one of: the positioning part being arranged on the supporting part, the positioning part being connected to the supporting part, and the positioning part being embedded in the supporting part.

According to the embodiments of the present disclosure, the preparing the supporting part and the positioning part according to the measurement region and the positioning portion so as to obtain the positioning apparatus may include the following operations.

The supporting part and the positioning part are prepared according to a structural parameter of the measurement region and a structural parameter of the positioning portion so as to obtain the positioning apparatus.

According to the embodiments of the present disclosure, the corresponding positioning part may be prepared according to the structural parameter of the positioning part. The corresponding supporting part may be prepared according to the structural parameter of the measurement region and/or the structural parameter of the positioning portion. A prepare method may include 3D printing or machining. The structural parameter may include a length, a width and a thickness, etc., of the positioning portion.

If the measurement region is the region on the outer side of the proximal knuckle of the index finger, the positioning portion may include the little finger, the ring finger, the middle finger and the index finger. Based on this, the positioning part may be prepared based on structural parameters of the first two knuckles of the little finger, the ring finger, the middle finger and the index finger, and the positioning part is the knuckle part.

If the measurement region is the forearm extensor side region, the positioning portion may include the little finger, the ring finger, the middle finger, the index finger, the palm and the elbow joint. Based on this, the knuckle part may be prepared based on structural parameters of the first two knuckles of the little finger, the ring finger, the middle finger and the index finger. The elbow joint part may be prepared based on a structural parameter of the arm and a structural parameter of the elbow joint. The positioning part may include the knuckle part and the elbow joint part described above. In addition, the positioning portion may further include the carpus. Based on this, the carpus part may be prepared based on a structural parameter of the carpus. Accordingly, the positioning part may further include the carpus part described above.

If the measurement region is the forehead region, the positioning portion may include the eye corner and the nose. Based on this, the eye corner part may be prepared based on a structural parameter of the eye corner. The nose part may be prepared based on a structural parameter of the nose. The positioning part may include the eye corner part and the nose part described above.

According to the embodiments of the present disclosure, in order to prevent the positioning apparatus from contacting the measurement region and affecting the state of the measurement region during the positioning process, a region corresponding to the measurement region on the supporting part is provided as a hollow region. A specific size of the hollow region may be determined based on the structural parameter of the measurement region.

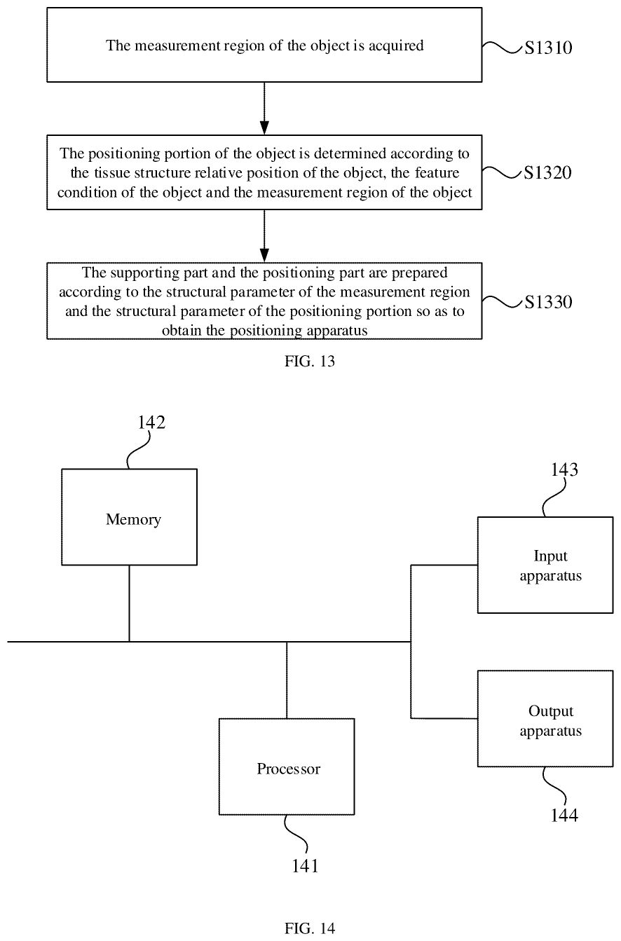

FIG. 13 is a flowchart of a method of preparing another positioning apparatus provided by the embodiments of the present disclosure. The embodiment may be applicable to ensure the measurement condition so as to improve the positioning accuracy and thereby improving the detection accuracy. As shown in FIG. 13, the method specifically includes the following operations.

In operation S1310, the measurement region of the object is acquired.

In operation S1320, the positioning portion of the object is determined according to the tissue structure relative position of the object, the feature condition of the object and the measurement region of the object.

In operation S1330, the supporting part and the positioning part are prepared according to the structural parameter of the measurement region and the structural parameter of the positioning portion so as to obtain the positioning apparatus.

According to the embodiments of the present disclosure, the relationship between the positioning part and the supporting part satisfies at least one of: the positioning part being arranged on the supporting part, the positioning part being connected to the supporting part, and the positioning part being embedded in the supporting part.

According to the technical solution of the embodiments of the present disclosure, since the corresponding positioning apparatus is prepared according to the measurement region, it has a strong practicability. Moreover, the preparation cost is low and the operation is simple and easy.

FIG. 14 is a schematic structural diagram of an electronic device provided by the embodiments of the present disclosure. The electronic device shown in FIG. 14 is merely an example, and should not limit functions and application scopes of the embodiments of the present disclosure. As shown in FIG. 14, the electronic device provided by the embodiments of the present disclosure includes a processor 141, a memory 142, an input apparatus 143 and an output apparatus 144. The electronic device may include one or more processors 141, and FIG. 14 shows one processor 141 as an example. The processor 141, the memory 142, the input apparatus 143 and the output apparatus 144 in the electronic device may be connected through a bus or in other manners, and FIG. 14 shows a connection through a bus as an example.

The memory 142 is a computer-readable storage medium, and may be used to store software programs, computer-executable programs and modules, such as program instructions/modules corresponding to the positioning method in the embodiments of the present disclosure. The processor 141 executes various functional applications and data processing by executing the software programs, instructions and modules stored in the memory 142, thereby implementing the positioning method applied to the electronic device provided by the embodiments of the present disclosure.

The memory 142 may mainly include a program storage area and a data storage area. The program storage area may store an operating system and an application program required by at least one function. The data storage area may store data generated by using the electronic apparatus. In addition, the memory 142 may include a high-speed random access memory, and may further include a non-transitory memory, such as at least one magnetic disk storage device, a flash memory device, or other non-transitory solid-state storage devices. In some embodiments, the memory 142 may further include a memory provided remotely with respect to the processor 141, and such remote memory may be connected to the electronic apparatus through a network. Examples of the above-mentioned network include, but are not limited to the Internet, an intranet, a local area network, a mobile communication network, and a combination thereof.

The input apparatus 143 may be configured to receive a numeric or character information input by a user, so as to generate a key signal input related to user settings and function control of the electronic apparatus. The output apparatus 144 may include a display electronic device such as a display screen.

Certainly, those skilled in the art may understand that the processor may also implement the technical solution of the positioning method applied to the electronic device provided by any embodiment of the present disclosure. For a hardware structure and a function of the electronic device, reference may be made to the content explanations of the embodiments.

The embodiments of the present disclosure further provide a computer-readable storage medium having computer programs stored thereon. When the programs are executed by a processor, a positioning method as provided in the embodiments of the present disclosure is implemented. The method may include the following operations.

The measurement region of the object is acquired. The positioning portion of the object is determined according to the measurement region. The positioning portion is positioned so as to position the measurement region and ensure that the measurement posture meets the predetermined condition.

The computer storage medium in the embodiments of the present disclosure may be any combination of one or more computer-readable mediums. The computer-readable medium may be a computer-readable signal medium or a computer-readable storage medium. Here, the computer-readable storage medium may be any tangible medium that contains or stores a program that may be used by or in combination with an instruction execution system, apparatus or device.

The computer-readable signal medium may contain a data signal propagated in a baseband or as a part of a carrier wave, in which a computer-readable program code is carried. Such propagated data signal may take various forms, including but not limited to an electromagnetic signal, an optical signal, or any suitable combination of thereof. The computer-readable signal medium may also be any computer-readable medium other than the computer-readable storage medium. The computer-readable medium may send, propagate, or transmit the program for use by or in combination with the instruction execution system, apparatus or device.

The program code contained on the computer-readable medium may be transmitted by any suitable medium, including but not limited to wireless, wired, optical cable, radio frequency, etc., or any suitable combination thereof.

Computer program codes used for performing operations of the present disclosure may be written in one or more programming languages, such as C language and Python, etc., or a combination thereof. The program codes may be executed on a computer or a server.

Certainly, for the computer-readable storage medium provided in the embodiments of the present disclosure, the computer-executable instructions thereof are not limited to the operations of the method described above, and may also be used to execute related operations of the positioning method for the electronic device provided in any embodiment of the present disclosure. For the introduction of the storage medium, reference may be made to the content explanation in the embodiments.

The specific embodiments of the present disclosure described above are not intended to limit the protection scope of the present disclosure. Any other corresponding changes and modifications made according to the technical concept of the present disclosure shall be included in the protection scope of the claims of the present disclosure.

1. A positioning method, comprising:
acquiring a measurement region of an object;
determining a positioning portion of the object according to the measurement region; and
positioning the positioning portion to position the measurement region and ensure that a measurement posture meets a predetermined condition.

2. The method according to claim 1, wherein the determining a positioning portion of the object according to the measurement region comprises:
determining the positioning portion of the object according to a tissue structure relative position of the object, a feature condition of the object and the measurement region of the object.

3. The method according to claim 1, wherein the positioning the positioning portion to position the measurement region and ensure that a measurement posture meets a predetermined condition comprises:
positioning the positioning portion through a positioning apparatus to position the measurement region and ensure that the measurement posture meets the predetermined condition.

4. The method according to claim 3, wherein the positioning apparatus comprises a supporting part and a positioning part; and a relationship between the positioning part and the supporting part satisfies at least one of: the positioning part being arranged on the supporting part; the positioning part being connected to the supporting part; and the positioning part being embedded in the supporting part; and
wherein the positioning the positioning portion through the positioning apparatus to position the measurement region and ensure that the measurement posture meets the predetermined condition comprises:
carrying the measurement region and/or the positioning portion through the supporting part; and
positioning the positioning portion through the positioning part to position the measurement region and ensure that the measurement posture meets the predetermined condition.

5. The method according to claim 4, wherein a region corresponding to the measurement region is a hollow region.

6. The method according to claim 4, wherein the positioning part comprises a knuckle part configured to position at least one of a little finger, a ring finger, a middle finger and an index finger; and the positioning part is embedded in the supporting part;
wherein the measurement region is a hand region, and the positioning portion comprises at least one of the little finger, the ring finger, the middle finger and the index finger; and
wherein the carrying the measurement region and/or the positioning portion through the supporting part comprises:
carrying the measurement region and the positioning portion through the supporting part; or
wherein the positioning part comprises a knuckle part configured to position at least one of a little finger, a ring finger, a middle finger and an index finger and an elbow joint part configured to position an elbow joint; and the positioning part is arranged on the supporting part or embedded in the supporting part;
wherein the measurement region comprises an arm region; and the positioning portion comprises the elbow joint, and at least one of the little finger, the ring finger, the middle finger and the index finger; and
wherein the carrying the measurement region and/or the positioning portion through the supporting part comprises:
carrying the measurement region and the positioning portion through the supporting part; or
wherein the positioning part comprises an eye corner part configured to position an eye corner and a nose part configured to position a nose; and the positioning part is connected to the supporting part;
wherein the measurement region is a head region; and the positioning portion comprises the eye corner and the nose; and
wherein the carrying the measurement region and/or the positioning portion through the supporting part comprises:
carrying the measurement region through the supporting part.

7. (canceled)

8. (canceled)

9. (canceled)

10. (canceled)

11. (canceled)

12. (canceled)

13. A positioning apparatus, comprising a supporting part and a positioning part, wherein a relationship between the positioning part and the supporting part satisfies at least one of: the positioning part being arranged on the supporting part; the positioning part being connected to the supporting part; and the positioning part being embedded in the supporting part;
wherein the supporting part is configured to carry a measurement region and/or a positioning portion of an object; and
wherein the positioning part is configured to position the positioning portion to position the measurement region and ensure that a measurement posture meets a predetermined condition.

14. The apparatus according to claim 13, wherein the supporting part comprises a hollow region corresponding to the measurement region.

15. The apparatus according to claim 14, wherein a positional relationship between the positioning portion and the measurement region meets a predetermined tissue structure relative position.

16. The apparatus according to claim 13, wherein the positioning part comprises a knuckle part configured to position at least one of a little finger, a ring finger, a middle finger and an index finger; and the positioning part is embedded in the supporting part;
wherein the measurement region is a hand region; and the positioning portion comprises at least one of the little finger, the ring finger, the middle finger and the index finger; and
wherein the supporting part is configured to carry the measurement region and the positioning portion.

17. The apparatus according to claim 16, wherein the hand region comprises a region on an outer side of a proximal knuckle of the index finger; and the positioning portion comprises the little finger, the ring finger, the middle finger and the index finger.

18. The apparatus according to claim 13, wherein the positioning part comprises a knuckle part configured to position at least one of a little finger, a ring finger, a middle finger and an index finger and an elbow joint part configured to position an elbow joint; and the positioning part is arranged on the supporting part or embedded in the supporting part;
wherein the measurement region is an arm region, and the positioning portion comprises the elbow joint, and at least one of the little finger, the ring finger, the middle finger and the index finger; and
wherein the supporting part is configured to carry the measurement region and the positioning portion.

19. The apparatus according to claim 18, wherein the arm region comprises a forearm extensor side; and the positioning portion comprises the little finger, the ring finger, the middle finger, the index finger and the elbow joint.

20. The apparatus according to claim 19, wherein the positioning part further comprises a carpus part configured to position a carpus; and the positioning portion further comprises the carpus.

21. The apparatus according to claim 13, wherein the positioning part comprises an eye corner part configured to position an eye corner and a nose part configured to position a nose; and the positioning part is connected to the supporting part;
wherein the measurement region is a head region, and the positioning portion comprises the eye corner and the nose; and
wherein the supporting part is configured to carry the measurement region.

22. The apparatus according to claim 21, wherein the head region comprises a forehead region.

23. An electronic device, comprising:
one or more processors;
a memory configured to store one or more programs;
wherein the one or more programs, when executed by the one or more processors, cause the one or more processors to implement the method according to claim 1.

24. A computer-readable storage medium having computer programs stored thereon, wherein the programs, when executed by a processor, implement the method according to claim 1.

25. A computer program product comprising computer programs, wherein the computer programs, when executed by a processor, implement the method according to claim 1.
